(19) United States
(12) Patent Application Publication
(10) Pub. No.: US 2018/0208563 A1
(43) Pub. Date: Jul. 26, 2018

(54) THERMODYNAMICALLY STABLE CRYSTAL MODIFICATION OF 2-CHLORO-3-(METHYLSULFANYL)-N-(1-METHYL-1H-TETRAZOL-5-YL)-4-(TRIFLUOROMETHYL)BENZAMIDE

(71) Applicant: BAYER CROPSCIENCE AKTIENGESELLSCHAFT, MONHEIM AM RHEIN (DE)

(72) Inventors: HARTMUT AHRENS, EGELSBACH (DE); ARNIM KÖHN, KLEIN-WINTERNHEIM (DE); CHRISTIAN WALDRAFF, BAD VILBEL (DE); BRITTA OLENIK, BOTTROP (DE); BIRGIT KEIL, DÜSSELDORF (DE)

(21) Appl. No.: 15/741,140

(22) PCT Filed: Jun. 30, 2016
(86) PCT No.: PCT/EP2016/065248

§ 371 Date: Dec. 29, 2017

(30) Foreign Application Priority Data
Jul. 3, 2015 (EP) 15175168.2

(51) Int. Cl. C07D 257/06; A01N 43/713

(52) U.S. Cl. CPC C07D 257/06; C07B 2200/13; A01N 43/713

(57) ABSTRACT

Thermodynamically stable crystal modification of 2-chloro-3-(methylsulfanyl)-N-(1-methyl-1H-tetrazol-5-yl)-4-(trifluoromethyl)benzamide

A thermodynamically stable crystal modification of the herbicidal active ingredient 2-chloro-3-(methylsulfanyl)-N-(1-methyl-1H-tetrazol-5-yl)-4-(trifluoromethyl)benzamide is described. This thermodynamically stable crystal modification has particular advantages in the stability of suspension formulations.

The invention relates to the technical field of crop protection compositions.

More specifically, it relates to a thermodynamically stable crystal modification of 2-chloro-3-(methylsulfanyl)-N-(1-methyl-1H-tetrazol-5-yl)-4-(trifluoromethyl)benzamide of the formula (I)

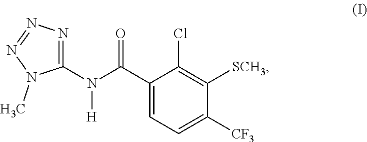 [SMILES: CSc1c(C(F)(F)F)ccc(C(=O)Nc2nnnn2C)c1Cl]

and to a method for the preparation thereof and use thereof as a herbicide. The compound of the formula (I) is referred to below as “benzamide” irrespective of its manifestation.

It is known that some organic compounds can occur in only one crystal structure, while others, so-called polymorphs, can occur in various crystal structures, see, for example, J. Bernstein, R. J. Davey, J. O. Henck, Angew. Chem. Int. Ed., 1999, 38, 3440-3461. For instance, two crystal structures of the herbicidal active ingredient sulcotrione are known from EP 1 314 724 A1.

The benzamide known for example from WO 2012/028579 A1 (example No. 4-638 in table 4 therein) has herbicidal properties and is suitable for the production of crop protection compositions which can be employed for weed control. However, it has been shown that the benzamide preparable according to the disclosure of WO 2012/028579 A1 is not suitable for the production of user-friendly administration forms. User-friendly administration forms are, for example, suspension formulations in which the benzamide is present finely ground in solid form. Testing in practice has shown that the benzamide preparable according to the disclosure of WO 2012/028579 A1 leads to crystal growth in suspension formulations and consequently to clumping and precipitation, so that the suspension formulation becomes unusable. The crystal growth can occur spontaneously or over a longer period and cannot be predicted.

It was therefore an object of the present invention to provide a modification of the benzamide which overcomes these disadvantages and is suitable for the preparation of a suspension formulation which is storage-stable over a prolonged period.

In the context of the present invention, it has been found that the benzamide occurs in a thermodynamically metastable and in a thermodynamically stable crystal modification.

In the context of the present invention, it has also been found that the thermodynamically stable crystal modification of the benzamide does not have the abovementioned disadvantages and is therefore particularly suitable for the preparation of suspension formulations such as suspoconcentrates, suspoemulsions and oil dispersions.

Moreover, the benzamide preparable according to the disclosure of WO 2012/028579 A1 has the disadvantage that it can be less readily worked up, filtered and purified. This disadvantage is overcome by the provision of the thermodynamically stable benzamide according to the invention.

The invention therefore relates to a thermodynamically stable crystal modification of benzamide 2-chloro-3-(methylsulfanyl)-N-(1-methyl-1H-tetrazol-5-yl)-4-(trifluoromethyl)benzamide.

In the following, the metastable crystal modification of the benzamide is referred to as “metastable crystal modification I” and the stable as “stable crystal modification II”. In the following, the terms “modification” and “crystal modification” are understood to be equivalent.

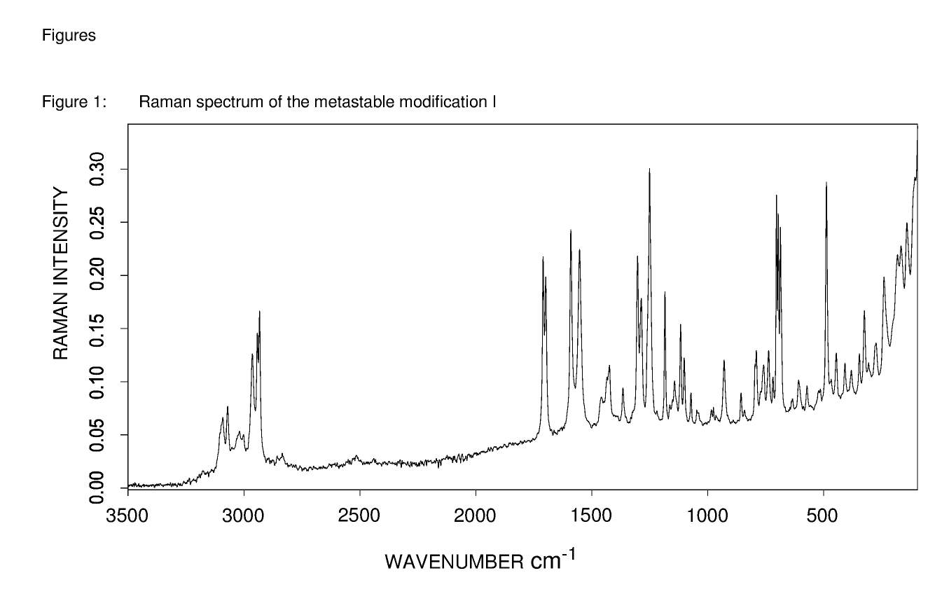

The metastable crystal modification I has a characteristic Raman spectrum, which is shown in FIG. 1, the values of the band maxima being given in wavenumbers in Table 1.

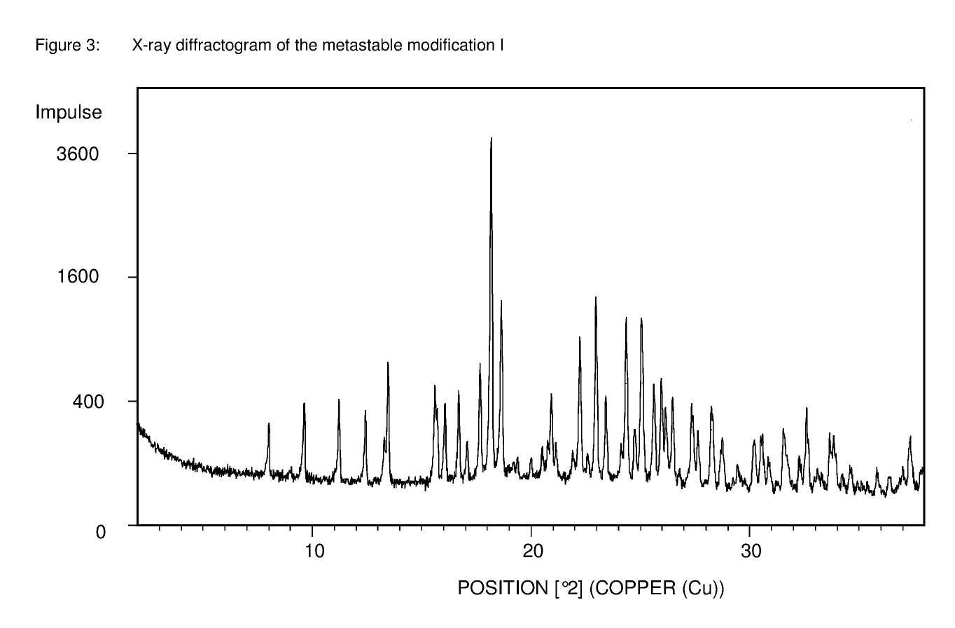

The X-ray powder diffractometry of the metastable crystal modification I shows characteristic peaks for this crystal modification which are given in Table 2. The corresponding X-ray diffractogram is shown in FIG. 3.

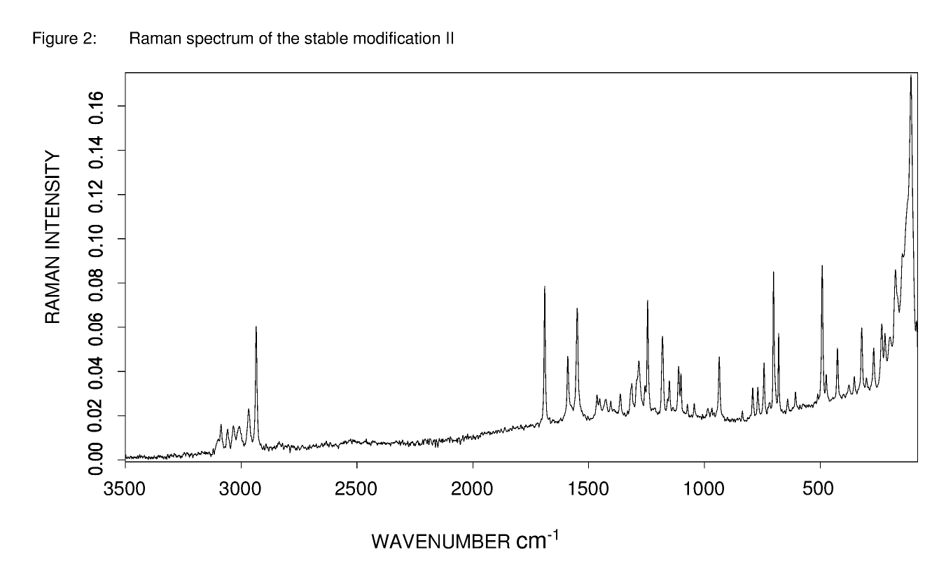

The stable crystal modification II also has a characteristic Raman spectrum, which is shown in FIG. 2, the values of the band maxima also being given in wavenumbers in Table 1.

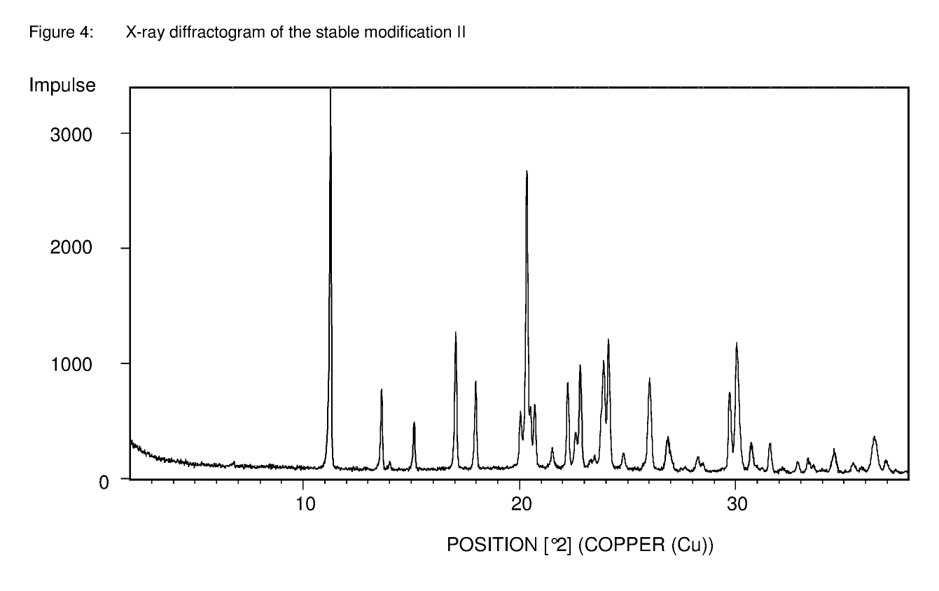

The X-ray powder diffractometry of the stable crystal modification II shows characteristic peaks for this crystal modification which are also given in Table 2. The corresponding X-ray diffractogram is shown in FIG. 4.

TABLE 1
Band maxima of the Raman spectra [cm⁻¹]

| Metastable modification I | Stable modification II |
|---|---|
| 3091 | 3086 |
| 3071 | 3058 |
| 3020 | 3034 |
| 3002 | 3008 |
| 2964 | 2968 |
| 2942 | 2935 |
| 2933 | 1690 |
| 2857 | 1590 |
| 2835 | 1549 |
| 1710 | 1464 |
| 1699 | 1452 |
| 1591 | 1425 |
| 1553 | 1404 |
| 1459 | 1363 |
| 1424 | 1313 |
| 1366 | 1283 |
| 1303 | 1257 |
| 1287 | 1245 |
| 1251 | 1181 |
| 1185 | 1151 |
| 1143 | 1112 |
| 1117 | 1101 |
| 1102 | 1074 |
| 1073 | 1044 |
| 1047 | 936 |
| 984 | 836 |
| 975 | 792 |
| 929 | 769 |
| 857 | 743 |
| 790 | 702 |
| 759 | 680 |
| 737 | 641 |
| 719 | 607 |
| 704 | 491 |
| 696 | 474 |
| 687 | 426 |
| 635 | 376 |
| 608 | 353 |
| 572 | 321 |
| 488 | 301 |
| 467 | 269 |
| 446 | 234 |
| 408 | 221 |
| 381 | 199 |
| 346 | 175 |
| 325 | 109 |
| 306 | |
| 274 | |
| 240 | |
| 182 | |
| 167 | |
| 141 | |
| 109 | |

### Measurement Conditions:

| | |
|---|---|
| Instrument | Bruker Raman RFS 100/S |
| Number of scans | 64 |
| Resolution | 2 cm⁻¹ |
| Laser Power | 50 mW |
| Laser wavelength | 1064 nm |

TABLE 2
X-ray powder diffractometry pattern
Peak maximum [2 theta]

| Metastable modification I | Stable modification II |
|---|---|
| 8.1 | 6.7 |
| 9.7 | 11.3 |
| 11.2 | 13.6 |
| 12.5 | 14.0 |
| 13.3 | 15.1 |
| 13.5 | 17.1 |
| 15.6 | 18.0 |
| 15.7 | 20.0 |
| 16.1 | 20.3 |
| 16.7 | 20.5 |
| 17.1 | 20.7 |
| 17.7 | 21.5 |
| 18.2 | 22.2 |
| 18.7 | 22.6 |
| 19.2 | 22.8 |
| 19.4 | 23.5 |
| 20.0 | 23.9 |
| 20.5 | 24.1 |
| 20.8 | 24.8 |
| 21.0 | 26.0 |
| 21.2 | 26.9 |
| 21.9 | 27.4 |
| 22.3 | 28.2 |
| 22.6 | 28.5 |
| 23.0 | 29.7 |
| 23.4 | 30.0 |
| 24.1 | 30.7 |
| 24.4 | 31.5 |
| 24.7 | 31.6 |
| 25.1 | 32.2 |
| 25.1 | 32.8 |
| 25.6 | 33.3 |
| 26.0 | 33.6 |
| 26.2 | 34.6 |
| 26.5 | 35.4 |
| 26.8 | 35.8 |
| 27.2 | 36.4 |
| 27.4 | 36.9 |
| 27.7 | |
| 28.3 | |
| 28.3 | |
| 28.7 | |
| 28.8 | |
| 29.3 | |
| 29.5 | |
| 29.6 | |
| 30.2 | |
| 30.5 | |
| 30.6 | |
| 30.9 | |
| 31.6 | |
| 31.7 | |
| 32.3 | |
| 32.6 | |
| 32.7 | |
| 33.0 | |
| 33.1 | |
| 33.3 | |
| 33.7 | |
| 33.9 | |
| 34.0 | |
| 34.3 | |
| 34.6 | |
| 34.7 | |
| 34.9 | |
| 35.9 | |
| 36.4 | |
| 36.9 | |
| 37.0 | |
| 37.3 | |
| 37.4 | |
| 37.9 | |

### Measurement Conditions:

| | |
|---|---|
| Scan axis | Gonio |
| Start position [°2Th.] | 2.0066 |
| End position [°2Th.] | 37.9906 |
| Anode, material | Cu |
| K-alpha1 [Å] | 1.54060 |
| K-alpha2 [Å] | 1.54443 |
| K-beta [Å] | 1.39225 |
| K-A2/K-A1 ratio | 0.50000 |
| Generator settings | 40 mA, 40 kV |
| Incident beam monochromator | focusing X-ray mirror |
| Spinning | Yes |

To determine the Raman spectra, an RFS 100/S FT-Raman from Bruker was used to record at least two spectra of 128 scans for each batch.

The single crystal X-ray structure analysis was determined by using a rotary anode M18X-HF with MoKα radiation from MACScience Co and a Bruker AXS SMART CCD 1000 detector. The data were processed with the programs SAINT-NT V 5.0 (data reduction, Bruker AXS) and SADABS (absorption correction, Bruker AXS). Structure solution and refinement was performed with SHELXTL-NT Version V5.1.

The benzamide of the formula (I) can be prepared per se by one of the methods described in WO 2012/028579 A1 for example. Depending on the type of solvent used in the final purification step and the temperature regime, the benzamide is usually obtained in amorphous form, in the form of the metastable crystal modification I described here or in a mixture of the amorphous form and the metastable crystal modification I.

The thermodynamically stable crystal modification II of the benzamide may be prepared for example in a general manner such that the benzamide obtainable according to WO 2012/028579 A1 is suspended and/or dissolved in a suitable solvent and it is treated at temperatures of 0° C. to 80° C. until quantitative conversion into the thermodynamically stable crystal modification II.

The invention therefore further relates to a method for preparing the thermodynamically stable crystal modification II of the benzamide, in which the crystal modification I of the benzamide is suspended and/or dissolved in solvents and it is treated at temperatures of 0° C. to 80° C. until quantitative conversion into the thermodynamically stable crystal modification II.

Suitable solvents which can be used in this method are, for example, lower alcohols such as methanol, ethanol, 2-propanol, or ketones such as acetone, 2-butanone, which can also be used in a mixture with water. Lower alcohols or ketones refer here to those compounds which have one to ten carbon atoms, preferably one to five carbon atoms. Further suitable solvents are benzene, toluene and chlorobenzene. Preference is given to toluene and mixtures of ethanol and water, particularly preferably toluene and a mixture of ethanol and water in the ratio 1:1.

The conversion to the thermodynamically stable crystal modification II is effected at temperatures less than 100° C., preferably at temperatures of 0° C. to 80° C., particularly preferably at temperatures of 20° C. to 80° C., especially preferably at temperatures of 20° C. to 40° C. The duration of the conversion depends on the temperature and type of solvent. In addition, the duration of the conversion depends on whether seed crystals of the crystal modification II are used. In general, the conversion to crystal modification II can be achieved directly, with complete dissolution of the crystals of crystal modification I at elevated temperature, by cooling crystallization to room temperature without using seed crystals. The cooling to room temperature is effected preferably with a cooling rate of less than 25° C., particularly preferably with a cooling rate of less than 20° C. The conversion of a suspension of crystal modification I can generally be brought about without the use of seed crystals within a period of 14 days. When seed crystals of crystal modification II are used in the conversion of a suspension, a treatment time of 24 to 48 hours is generally sufficient in order to achieve a quantitative conversion of the crystals to the crystal modification II.

The resulting crystals of crystal modification II are finally separated off and are dried to constant weight by removing the solvent at room temperature or elevated temperature.

The stable crystal modification II can also be obtained from the crystal modification I or the amorphous form by grinding under high pressure. A suitable pressure is a pressure of at least 5 bar.

The crystal modification II, due to its stability, is outstandingly suitable for the preparation of formulations, especially suspension formulations, of crop protection compositions. Accordingly, the invention also provides crop protection compositions which comprise crystal modification II of the benzamide alone or as a mixture with auxiliaries and carriers, and also as a mixture with other active ingredients. The invention also includes mixtures of crystal modification II of the benzamide with crystal modification I of the benzamide, for example those which arise at any point during the conversion process according to the invention of crystal modification I into crystal modification II. Preference is given to an active ingredient quality with more than 80% by weight of crystal modification II of the benzamide, particularly preferably with more than 90% by weight, especially preferably with more than 95% by weight and most preferably with more than 98% by weight.

The benzamide of the stable crystal modification II is optionally mixed with one or more other herbicides. Such mixtures also profit from the advantageous properties of the crystal modification II according to the invention.

Owing to its stability, the stable crystal modification II of the benzamide is suitable in general terms as starting material for the preparation of any plant protection formulations comprising this benzamide, even when the benzamide is no longer in this form following formulation but rather in dissolved form.

The invention therefore also relates to a method for preparing the plant protection formulations comprising the benzamide which employ the stable crystal modification II of the benzamide and also plant protection formulations comprising this benzamide obtained from the stable crystal modification II of the benzamide. By using the stable crystal modification II, the safety of the preparations of the benzamide is increased and therefore the risk of incorrect dosages decreases.

The stable crystal modification II of the benzamide can be converted in a known manner to the customary formulations, such as suspension concentrates, colloidal concentrates, dispersible concentrates, emulsifiable concentrates (emulsion concentrates), seed-dressing emulsions, seed-dressing suspensions, granules, microgranules, suspoemulsions, oil dispersions, water-soluble granules, water-soluble concentrates and water-dispersible granules, using suitable auxiliaries and carriers or solvents. In this connection, the active ingredient should be present in a concentration of approximately 0.5 to 90% by weight of the total mixture, i.e. in amounts which are sufficient in order to achieve the dosage level required. The formulations are prepared, for example, by extending the stable crystal modification II of the benzamide with solvents and/or carriers, optionally using emulsifiers and/or dispersants, and/or other auxiliaries, for example penetrants.

Application is effected in a customary manner, by contacting the unwanted plants and/or their habitat with the active ingredient or formulations thereof.

Moreover, the thermodynamically stable crystal modification II of the benzamide can be readily processed, filtered and purified.

The benzamide in the stable crystal modification II shows excellent herbicidal activity against representatives of the group of both monocotyledonous and dicotyledonous plants. Examples here include:

Dicotyledonous Plants of the Genera:

Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Monocotyledonous Plants of the Genera:

Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

The invention therefore also relates to the use of the stable crystal modification II of the benzamide for preparing a plant protection composition for the treatment of weed infestation.

The stable crystal modification II of the benzamide according to the invention is suitable, due to its compatibility with crop plants for controlling unwanted plants in crops of, for example, wheat, barley, oats, rye, rice, maize, sugar beet, sugar cane, cotton and soya, in particular in wheat, barley, oats and rye.

All plants and plant parts can be treated in accordance with the invention. Plants in this context are understood to include all plants and plant populations, such as desired and unwanted wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights. Plant parts are to be understood as meaning all above-ground and below-ground parts and organs of plants, such as shoot, leaf, flower and root, examples which may be mentioned being leaves, needles, stems, trunks, flowers, fruit bodies, fruits and seeds and also roots, tubers and rhizomes. Plant parts also include harvested material and vegetative and generative propagation material, for example cuttings, tubers, rhizomes, slips and seeds.

Treatment according to the invention of the plants and plant parts with the crystal modification II of the benzamide according to the invention is carried out directly or by allowing the compounds to act on the surroundings, habitat or storage space by the customary treatment methods, for example by immersion, spraying, evaporation, fogging, scattering or painting on.

The crystal modification II of the benzamide according to the invention, as already explained above, may be converted into the customary formulations such as solutions, emulsions, wettable powders, suspensions, powders, dusts, pastes, soluble powders, granules, suspension-emulsion concentrates, natural and synthetic materials impregnated with active ingredient, and microencapsulations in polymeric materials.

These formulations are produced in a known manner, for example by mixing the active compounds with extenders, that is, liquid solvents and/or solid carriers, optionally with the use of surfactants, that is to say, emulsifiers and/or dispersants, and/or foam formers.

If the extender used is water, it is also possible to use, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, or else water.

Suitable solid carriers are for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are for example crushed and fractionated natural rocks, such as calcite, marble, pumice, sepiolite, dolomite and synthetic granules of inorganic and organic meals, and granules of organic material, such as sawdust, coconut shells, maize cobs and tobacco stalks; suitable emulsifiers and/or foam formers are for example nonionogenic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and protein hydrolysates; suitable dispersants are for example lignosulfite waste liquors and methylcellulose.

In the formulations it is possible to use tackifiers such as carboxymethyl cellulose, natural and synthetic polymers in the form of powders, granules or latexes, such as gum arabic, polyvinyl alcohol and polyvinyl acetate, or else natural phospholipids such as cephalins and lecithins and synthetic phospholipids. Further additives may be mineral and vegetable oils.

It is possible to use colorants such as inorganic pigments, for example iron oxide, titanium oxide and Prussian blue, and organic colorants such as alizarin colorants, azo-colorants and metal phthalocyanine colorants, and trace nutrients such as salts of iron, manganese, boron, copper, cobalt, molybdenum and zinc.

In general, the formulations comprise between 0.1 and 95 percent by weight of the active ingredient in the form of crystal modification II according to the invention, preferably between 0.5 and 90%.

For controlling weeds, crystal modification II of the benzamide according to the invention, as such or in their formulations, can also be used as mixtures with known herbicides and/or substances which improve compatibility with crop plants (“safeners”), finished formulations or tank mixes being possible. Also possible are mixtures with weedkillers comprising one or more known herbicides and a safener.

Possible components for the mixtures are known herbicides, for example acetochlor, acifluorfen (-sodium), aclonifen, alachlor, alloxydim (-sodium), ametryne, amicarbazone, amidochlor, amidosulfuron, anilofos, asulam, atrazine, azafenidin, azimsulfuron, beflubutamid, benazolin (-ethyl), benfuresate, bensulfuron (-methyl), bentazon, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxydim, carbetamide, carfentrazone (-ethyl), chlomethoxyfen, chloramben, chloridazon, chlorimuron (-ethyl), chlornitrofen, chlorsulfuron, chlortoluron, cinidon (-ethyl), cinmethylin, cinosulfuron, clefoxydim, clethodim, clodinafop (-propargyl), clomazone, clomeprop, clopyralid, clopyrasulfuron (-methyl), cloransulam (-methyl), cumyluron, cyanazine, cybutryne, cycloate, cyclosulfamuron, cycloxydim, cyhalofop (-butyl), 2,4-D, 2,4-DB, desmedipham, diallate, dicamba, dichlorprop (—P), diclofop (-methyl), diclosulam, diethatyl (-ethyl), difenzoquat, diflufenican, diflufenzopyr, dimefuron, dimepiperate, dimethachlor, dimethametryn, dimethenamid, dimexyflam, dinitramine, diphenamid, diquat, dithiopyr, diuron, dymron, epropodan, EPTC, esprocarb, ethalfluralin, ethametsulfuron (-methyl), ethofumesate, ethoxyfen, ethoxysulfuron, etobenzanid, fenoxaprop (—P-ethyl), fentrazamide, flamprop (-isopropyl, -isopropyl-L, -methyl), flazasulfuron, florasulam, fluazifop (—P-butyl), fluazolate, flucarbazone (-sodium), flufenacet, flumetsulam, flumiclorac (-pentyl), flumioxazin, flumipropyn, flumetsulam, fluometuron, fluorochloridone, fluoroglycofen (-ethyl), flupoxam, flupropacil, flupyrsulfuron (-methyl, -sodium), flurenol (-butyl), fluridone, fluroxypyr (-butoxypropyl, -meptyl), flurprimidol, flurtamone, fluthiacet (-methyl), fluthiamide, fomesafen, foramsulfuron, glufosinate (-ammonium), glyphosate (-isopropylammonium), halosafen, haloxyfop (-ethoxyethyl, —P-methyl), hexazinone, imazamethabenz (-methyl), imazamethapyr, imazamox, imazapic, imazapyr, imazaquin, imazethapyr, imazosulfuron, iodosulfuron (-methyl, -sodium), ioxynil, isopropalin, isoproturon, isouron, isoxaben, isoxachlortole, isoxaflutole, isoxapyrifop, lactofen, lenacil, linuron, MCPA, mecoprop, mefenacet, mesosulfuron (-methyl, -sodium), mesotrione, metamitron, metazachlor, methabenzthiazuron, metobenzuron, metobromuron, (alpha-) metolachlor, metosulam, metoxuron, metribuzin, metsulfuron (-methyl), molinate, monolinuron, naproanilide, napropamide, neburon, nicosulfuron, norflurazon, orbencarb, oryzalin, oxadiargyl, oxadiazon, oxasulfuron, oxaziclomefone, oxyfluorfen, paraquat, pelargonic acid, pendimethalin, pendralin, pentoxazone, phenmedipham, picolinafen, pinoxaden, piperophos, pretilachlor, primisulfuron (-methyl), profluazol, prometryn, propachlor, propanil, propaquizafop, propisochlor, propoxycarbazone (-sodium), propyzamide, prosulfocarb, prosulfuron, pyraflufen (ethyl), pyrasulfotole, pyrazogyl, pyrazolate, pyrazosulfuron (-ethyl), pyrazoxyfen, pyribenzoxim, pyributicarb, pyridate, pyridatol, pyriftalid, pyriminobac (-methyl), pyrithiobac (-sodium), quinchlorac, quinmerac, quinoclamine, quizalofop (—P-ethyl, —P-tefuryl), rimsulfuron, sethoxydim, simazine, simetryn, sulfentrazone, sulfometuron (-methyl), sulfosate, sulfosulfuron, tebutam, tebuthiuron, tepraloxydim, terbuthylazine, terbutryn, thenylchlor, thiafluamide, thiazopyr, thidiazimin, thifensulfuron (-methyl), thiobencarb, tiocarbazil, tralkoxydim, triallate, triasulfuron, tribenuron (-methyl), triclopyr, tridiphane, trifluralin, trifloxysulfuron, triflusulfuron (-methyl), tritosulfuron.

Furthermore, known safeners are suitable for the mixtures, for example: AD-67, BAS-145138, benoxacor, cloquintocet (-mexyl), cyometrinil, cyprosulfamide, 2,4-D, DKA-24, dichlormid, dymron, fenclorim, fenchlorazole (-ethyl), flurazole, fluxofenim, furilazole, isoxadifen (-ethyl), MCPA, mecoprop (—P), mefenpyr (-diethyl), MG-191, oxabetrinil, PPG-1292, R-29148.

A mixture with other known active compounds, such as fungicides, insecticides, acaricides, nematicides, bird repellents, plant nutrients and agents which improve soil structure, is also possible.

The crystal modification II of the benzamide according to the invention can be used as such, in the form of its formulations or in the use forms prepared therefrom by further dilution, such as ready-to-use solutions, suspensions, emulsions, powders, pastes and granules. Application is accomplished in a customary manner, for example by watering, spraying, atomizing or broadcasting.

The crystal modification II of the benzamide according to the invention can be applied both before and after emergence of the plants. It can also be incorporated into the soil before sowing.

The amount of active compound used can vary within a relatively wide range. It depends essentially on the nature of the desired effect. In general, the amounts used are between 1 g and 1 kg of active ingredient per hectare of soil surface, preferably between 5 g and 500 g per ha.

As already mentioned above, it is possible to treat all plants and their parts in accordance with the invention. In a preferred embodiment, wild plant species and plant cultivars, or those obtained by conventional biological breeding techniques, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are to be understood as meaning plants having certain properties (“traits”) which have been obtained by conventional breeding, by mutagenesis or by recombinant DNA techniques. They may be cultivars, biotypes and genotypes.

Depending on the plant species or plant cultivars, and the location and growth conditions (soils, climate, vegetation period, diet) thereof, the treatment according to the invention may also result in superadditive (“synergistic”) effects. Thus, for example, reduced application rates and/or a widening of the activity spectrum and/or an increase in the activity of the substances and compositions to be used according to the invention—also in combination with other agrochemical active ingredients —, better crop plant growth, increased tolerance of the crop plants to high or low temperatures, increased tolerance of the crop plants to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The preferred transgenic plants or plant cultivars (those obtained by genetic engineering) which are to be treated in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher harvest yields, higher quality and/or higher nutritional value of the harvested products, better capability for storage and/or processability of the harvested products. Further and particularly emphasized examples of such properties are an improved defense of the plants against animal and microbial pests, such as against insects, mites, phytopathogenic fungi, bacteria and/or viruses, and also increased tolerance of the plants to certain herbicidally active compounds. Examples of transgenic plants are the important crop plants, such as cereals (wheat, rice), soya beans, potatoes, cotton, oilseed rape and also in particular maize, and also fruit plants (with the fruits being apples, pears, citrus fruits and grapes), and particular emphasis is given particularly to maize, but also to soya beans, potatoes, cotton and oilseed rape. Traits that are particularly emphasized are the increased defence of the plants against insects, by means of toxins which form in the plants, especially those generated in the plants by the genetic material from Bacillus thuringiensis (e.g. by the genes CryIA(a), CryIA(b), CryIA(c), CryIIA, CryIIIA, CryIIIB2, Cry9c, Cry2Ab, Cry3Bb and CryIF, and also combinations thereof) (hereinafter “Bt plants”). Traits that are also particularly emphasized are the improved defense of plants against fungi, bacteria and viruses by systemic acquired resistance (SAR), systemin, phytoalexins, elicitors and also resistance genes and correspondingly expressed proteins and toxins. Traits that are additionally particularly emphasized are the increased tolerance of the plants to certain herbicidally active compounds, for example imidazolinones, sulfonylureas, glyphosate or phosphinothricin (for example the “PAT” gene). The genes which impart the desired properties (“traits”) in question may also be present in combinations with one another in the transgenic plants. Examples of “Bt plants” which may be mentioned are particularly maize varieties but also cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants include particularly maize varieties but also cotton varieties and soya varieties which are sold under the commercial names Roundup Ready® (tolerance to glyphosate e.g. maize, cotton, soya beans), Liberty Link® (tolerance to phosphinothricin, e.g. oilseed rape), IMI® (tolerance to imidazolinones) and STS® (tolerance to sulfonylureas, e.g. maize). Herbicide-resistant plants (bred conventionally for herbicide tolerance) also include the varieties sold under the Clearfield® name (e.g. maize). Of course, these statements also apply to plant cultivars which have these genetic traits or genetic traits which are yet to be developed and will be developed and/or marketed in the future.

### WORKING EXAMPLES

Preparation of the thermodynamically stable crystal modification II

Variant 1: 0.1 g of the benzamide prepared in accordance with the method disclosed in WO 2012/028579 A1 were suspended in 0.5 ml of ethanol:water (1:1). The suspension is shaken at 25° C. and stirred at 20 to 25° C. for 24 hours. A further 0.5 ml of ethanol:water (1:1) are then added and the suspension is stirred at 20 to 25° C. for 8 days. The suspension is filtered and the residue is allowed to stand open at room temperature until the solvent has evaporated. This affords the benzamide in the thermodynamically stable crystal modification II.

Variant 2: 0.1 g of the benzamide prepared in accordance with the method disclosed in WO 2012/028579 A1 were suspended in 0.25 ml of toluene. The suspension is shaken at 25° C. and stirred at 20 to 25° C. for 24 hours. A further 0.25 ml of toluene are then added and the suspension is stirred at 20 to 25° C. for 6 days. A further 0.25 ml of toluene are again added and the suspension is stirred at 20 to 25° C. for 2 days. The suspension is allowed to stand open at room temperature until the solvent has evaporated. This affords the benzamide in the thermodynamically stable crystal modification II.

### Stability Tests

An oil dispersion of the benzamide of the crystal modification II, compared to an oil dispersion of the benzamide prepared according to the methods disclosed in WO 2012/028579 A1, shows no signs of clumping and precipitation even after several weeks of storage.

1. A thermodynamically stable crystal modification of 2-chloro-3-(methylsulfanyl)-N-(1-methyl-1H-tetrazol-5-yl)-4-(trifluoromethyl)benzamide, wherein the crystal modification
a) has a Raman spectrum with band maxima [cm⁻¹] of:

| | |
|---|---|
| 3086 | 1044 |
| 3058 | 936 |
| 3034 | 836 |
| 3008 | 792 |
| 2968 | 769 |
| 2935 | 743 |
| 1690 | 702 |
| 1590 | 680 |
| 1549 | 641 |
| 1464 | 607 |
| 1452 | 491 |
| 1425 | 474 |
| 1404 | 426 |
| 1363 | 376 |
| 1313 | 353 |
| 1283 | 321 |
| 1257 | 301 |
| 1245 | 269 |
| 1181 | 234 |
| 1151 | 221 |
| 1112 | 199 |
| 1101 | 175 |
| 1074 | 109 |

and
b) has an X-ray powder diffractometry pattern with the following peaks, specified in degrees 2 theta:

| | |
|---|---|
| 6.7 | 26.0 |
| 11.3 | 26.9 |
| 13.6 | 27.4 |
| 14.0 | 28.2 |
| 15.1 | 28.5 |
| 17.1 | 29.7 |
| 18.0 | 30.0 |
| 20.0 | 30.7 |
| 20.3 | 31.5 |
| 20.5 | 31.6 |
| 20.7 | 32.2 |
| 21.5 | 32.8 |
| 22.2 | 33.3 |
| 22.6 | 33.6 |
| 22.8 | 34.6 |
| 23.5 | 35.4 |
| 23.9 | 35.8 |
| 24.1 | 36.4 |
| 24.8 | 36.9 |

2. An herbicidal agent comprising a content of the thermodynamically stable crystal modification of 2-chloro-3-(methylsulfanyl)-N-(1-methyl-1H-tetrazol-5-yl)-4-(trifluoromethyl)benzamide as claimed in claim 1 and one or more extenders and/or surface-active auxiliaries.

3. An herbicidal agent comprising the thermodynamically stable crystal modification of 2-chloro-3-(methylsulfanyl)-N-(1-methyl-1H-tetrazol-5-yl)-4-(trifluoromethyl)benzamide as claimed in claim 1 and a metastable crystal modification, wherein the 2-chloro-3-(methylsulfanyl)-N-(1-methyl-1H-tetrazol-5-yl)-4-(trifluoromethyl)benzamide comprises more than 90% by weight in the stable crystal modification.

4. The herbicidal agent as claimed in claim 3, wherein the 2-chloro-3-(methylsulfanyl)-N-(1-methyl-1H-tetrazol-5-yl)-4-(trifluoromethyl)benzamide comprises more than 95% by weight in the stable crystal modification.

5. The herbicidal agent as claimed in claim 3, wherein the 2-chloro-3-(methylsulfanyl)-N-(1-methyl-1H-tetrazol-5-yl)-4-(trifluoromethyl)benzamide comprises more than 98% by weight in the stable crystal modification.

6. A product comprising a thermodynamically stable crystal modification of 2-chloro-3-(methylsulfanyl)-N-(1-methyl-1H-tetrazol-5-yl)-4-(trifluoromethyl)benzamide as claimed in claim 1 or an agent comprising said modification, for controlling unwanted plants.

7. A method for controlling one or more unwanted plants, comprising allowing the thermodynamically stable crystal modification of 2-chloro-3-(methylsulfanyl)-N-(1-methyl-1H-tetrazol-5-yl)-4-(trifluoromethyl)benzamide as claimed in claim 1 or an agent comprising said modification to act on an unwanted plant and/or a habitat thereof.

8. The method as claimed in claim 7 for control of harmful plants in one or more monocotyledonous plant crops.

9. The method as claimed in claim 7 in which the plant is genetically modified or has been obtained by mutation-selection.
